(19) United States
(12) Patent Application Publication
(10) Pub. No.: US 2016/0347848 A1
(43) Pub. Date: Dec. 1, 2016

(54) THERAPEUTIC COMBINATIONS AND METHODS FOR TREATING NEOPLASIA

(71) Applicant: MedImmune Limited, Cambridge (GB)

(72) Inventors: Scott A. HAMMOND, Gaithersburg, MD (US); Kathleen Ann MULGREW, Gaithersburg, MD (US); Michael OBERST, Gaithersburg, MD (US); Edward BRADLEY, Gaithersburg, MD (US); Ross A. STEWART, Cambridge (GB)

(73) Assignee: MedImmune Limited, Cambridge (GB)

(21) Appl. No.: 15/149,927

(22) Filed: May 9, 2016

Related U.S. Application Data
(60) Provisional application No. 62/167,625, filed on May 28, 2015.

(51) Int. Cl. C07K 16/28; C07K 16/30

(52) U.S. Cl. CPC C07K 16/2878; C07K 16/2827; C07K 16/2818; C07K 16/30; C07K 16/3069; C07K 16/3015; C07K 16/3038; C07K 16/3023; C07K 2317/76; C07K 2317/21

(57) ABSTRACT

Provided herein are methods of treating a solid tumor comprising administering an effective amount of MEDI4736 or an antigen-binding fragment thereof, tremelimumab or an antigen-binding fragment thereof, and MEDI6383.

### SEQUENCE LISTING

The instant application contains a Sequence Listing which has been submitted electronically in ASCII format and is hereby incorporated by reference in its entirety. Said ASCII copy, created on Apr. 14, 2016, is named BTO-200US1_SL.txt and is 34,074 bytes in size.

### BACKGROUND OF THE INVENTION

Cancer continues to be a major global health burden. Despite progress in the treatment of cancer, there continues to be an unmet medical need for more effective and less toxic therapies, especially for those patients with advanced disease or cancers that are resistant to existing therapeutics.

The role of the immune system, in particular T cell-mediated cytotoxicity, in tumor control is well recognized. There is mounting evidence that T cells control tumor growth and survival in cancer patients, both in early and late stages of the disease. However, tumor-specific T-cell responses are difficult to mount and sustain in cancer patients.

T cell pathways receiving significant attention to date signal through cytotoxic T lymphocyte antigen-4 (CTLA-4, CD152), programmed death ligand 1 (PD-L1, also known as B7-H1 or CD274), and OX40 (CD134; TNFRSF4).

CTLA4 is expressed on activated T cells and serves as a co-inhibitor to keep T cell responses in check following CD28-mediated T cell activation. CTLA4 is believed to regulate the amplitude of the early activation of naïve and memory T cells following TCR engagement and to be part of a central inhibitory pathway that affects both antitumor immunity and autoimmunity. CTLA4 is expressed exclusively on T cells, and the expression of its ligands CD80 (B7.1) and CD86 (B7.2), is largely restricted to antigen-presenting cells, T cells, and other immune mediating cells. Antagonistic anti-CTLA4 antibodies that block the CTLA4 signaling pathway have been reported to enhance T cell activation. One such antibody, ipilimumab, was approved by the FDA in 2011 for the treatment of metastatic melanoma. Another anti-CTLA4 antibody, tremelimumab, was tested in phase III trials for the treatment of advanced melanoma, but did not significantly increase the overall survival of patients compared to the standard of care (temozolomide or dacarbazine) at that time.

PD-L1 is also part of a complex system of receptors and ligands that are involved in controlling T-cell activation. In normal tissue, PD-L1 is expressed on T cells, B cells, dendritic cells, macrophages, mesenchymal stem cells, bone marrow-derived mast cells, as well as various nonhematopoietic cells. Its normal function is to regulate the balance between T-cell activation and tolerance through interaction with its two receptors: programmed death 1 (also known as PD-1 or CD279) and CD80 (also known as B7-1 or B7.1). PD-L1 is also expressed by tumors and acts at multiple sites to help tumors evade detection and elimination by the host immune system. PD-L1 is expressed in a broad range of cancers with a high frequency. In some cancers, expression of PD-L1 has been associated with reduced survival and unfavorable prognosis. Antibodies that block the interaction between B7-H1 and its receptors are able to relieve PD-L1-dependent immunosuppressive effects and enhance the cytotoxic activity of antitumor T cells in vitro. MEDI4736 is a human monoclonal antibody directed against human PD-L1 that is capable of blocking the binding of PD-L1 to both the PD-1 and CD80 receptors.

OX40 is a tumor necrosis factor receptor (TNFR) found primarily on activated CD4+ and CD8+ T cells, regulatory T cells (Treg), and natural killer (NK) cells. Signaling through OX40 on activated CD4+ and CD8+ T cells leads to enhanced cytokine production, granzyme and perforin release, and expansion of effector and memory T-cell pools. In addition, OX40 signaling on Treg cells inhibits expansion of Tregs, shuts down the induction of Tregs, and blocks Treg-suppressive function.

Despite the significant progress made over the past decade in developing strategies for combatting cancer and other diseases, patients with advanced, refractory and metastatic disease have limited clinical options. Chemotherapy, irradiation, and high dose chemotherapy have become dose limiting. There remains a substantial unmet need for new less-toxic methods and therapeutics that have better therapeutic efficacy, longer clinical benefit, and improved safety profiles, particularly for those patients with advanced disease or cancers that are resistant to existing therapeutics.

### SUMMARY OF THE INVENTION

In one aspect, the invention provides a method of treating a solid tumor in a subject (e.g., a human subject), involving administering an anti-PD-L1 antibody (e.g., MEDI4736) or an antigen-binding fragment thereof, an anti-CTLA-4 antibody (e.g., tremelimumab) or an antigen-binding fragment thereof, and an OX40 agonist (e.g., MEDI6383) to the subject.

In another aspect, the invention provides a method of treating a solid tumor in a subject (e.g., a human subject), comprising administering MEDI4736 or an antigen-binding fragment thereof, tremelimumab or an antigen-binding fragment thereof, and MEDI6383 to the subject.

In another aspect, the invention provides a pharmaceutical composition containing an effective amount of an anti-PD-L1 antibody (e.g., MEDI4736) or an antigen-binding fragment thereof, an anti-CTLA-4 antibody (e.g., tremelimumab) or an antigen-binding fragment thereof, and an OX40 agonist (e.g., MEDI6383) and a pharmaceutically acceptable excipient.

In another aspect, the invention provides a pharmaceutical composition containing an effective amount of MEDI4736 or an antigen-binding fragment thereof, tremelimumab or an antigen-binding fragment thereof, and MEDI6383 or an active fragment thereof and a pharmaceutically acceptable excipient.

In another aspect, the invention provides a kit containing a pharmaceutical composition according to any other aspect delineated herein and instructions for the treatment of cancer (e.g., a method according to any other aspect delineated herein).

In various embodiments of any aspect delineated herein, the OX40 agonist is one or more of an OX40 ligand fusion protein (e.g., MEDI6383) or an anti-OX40 antibody.

In various embodiments of any aspect delineated herein, the anti-PD-L1 antibody is MEDI4736.

In various embodiments of any aspect delineated herein, the anti-CTLA-4 antibody is tremelimumab.

In various embodiments of any aspect delineated herein, the administrations increase survival. In various embodiments, the administrations result in an increase in survival as compared to the administration of MEDI4736 alone, tremelimumab alone, or MED6383 alone. In further embodiments, the administrations result in an increase in survival as compared to the administration of MEDI4736 and tremelimumab, MEDI4736 and MEDI6383, and tremelimumab and MEDI6383.

In various embodiments of any aspect delineated herein, the administrations decrease tumor volume. In various embodiments, the administrations result in a decrease in tumor volume as compared to the administration of MEDI4736 alone, tremelimumab alone, or MED6383 alone. In further embodiments, the administrations result in a decrease in tumor volume as compared to the administration of MEDI4736 and tremelimumab, MEDI4736 and MEDI6383, and tremelimumab and MEDI6383.

In various embodiments of any aspect delineated herein, the administration of anti-PD-L1 antibody (e.g., MEDI4736) or an antigen-binding fragment thereof is by intravenous infusion. In various embodiments of any aspect delineated herein, the administration of anti-CTLA-4 antibody (e.g., tremelimumab) or an antigen-binding fragment thereof is by intravenous infusion. In various embodiments of any aspect delineated herein, the administration of an OX40 agonist (e.g., MEDI6383) or an active fragment thereof is by intravenous infusion. In various embodiments of any aspect delineated herein, the pharmaceutical composition is formulated for intravenous administration.

In various embodiments of any aspect delineated herein, the solid tumor is one or more of ovarian cancer, breast cancer (e.g., triple negative breast cancer), colorectal cancer, prostate cancer, cervical cancer, uterine cancer, testicular cancer, bladder cancer, head and neck cancer, melanoma, pancreatic cancer, renal cell carcinoma, lung cancer, or non-small cell lung cancer (e.g., squamous or non-squamous non-small cell lung cancer). In various embodiments of any aspect delineated herein, the subject is a human patient.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “anti-tumor activity” is meant any biological activity that reduces or stabilizes the proliferation or survival of a tumor cell. In one embodiment, the anti-tumor activity is an anti-tumor immune response.

By “immunomodulatory agent” is meant an agent that enhances an immune response (e.g., anti-tumor immune response). Exemplary immunomodulatory agents of the invention include antibodies, such as an anti-CTLA-4 antibody, an anti-PD-L1 antibody, and fragments thereof, as well as proteins, such as OX40 ligand fusion protein, or fragments thereof. In one embodiment, the immunomodulatory agent is an immune checkpoint inhibitor.

By “OX40 polypeptide” is meant a polypeptide or fragment thereof having at least about 85% amino acid identity to NCBI Accession No. NP_003318. OX40 is a member of the TNFR-superfamily of receptors that is expressed on the surface of antigen-activated mammalian CD4+ and CD8+T lymphocytes. See, for example, Paterson et al., Mol Immunol 24, 1281-1290 (1987); Mallett et al., EMBO J 9, 1063-1068 (1990); and Calderhead et al., J Immunol 151, 5261-5271 (1993)). OX40 is also referred to as CD134, ACT-4, and ACT35. OX40 receptor sequences are known in the art and are provided, for example, at GenBank Accession Numbers: AAB33944 or CAE11757.

An exemplary human OX40 amino acid sequence is provided below (SEQ ID NO: 18):

| | |
|---|---|
| 1 | mcvgarrlgr gpcaallllg lglstvtglh cvgdtypsnd rcchecrpgn gmvsrcsrsq |
| 61 | ntvcrpcgpg fyndvvsskp ckpctwcnlr sgserkqlct atqdtvcrcr agtqpldsyk |
| 121 | pgvdcapcpp ghfspgdnqa ckpwtnctla gkhtlqpasn ssdaicedrd ppatqpqetq |
| 181 | gpparpitvq pteawprtsq gpstrpvevp ggravaailg lglvlgllgp laillalyll |
| 241 | rrdqrlppda hkppgggsfr tpiqeeqada hstlaki |

By “OX40 ligand” is meant a polypeptide or fragment thereof having at least about 85% amino acid identity to NCBI Accession No. NP_003317 and that specifically binds the OX40 receptor. See, for example, Baum P. R., et al. EMBO J. 13:3992-4001 (1994)). The term OX40L includes the entire OX40 ligand, soluble OX40 ligand, and fusion proteins comprising a functionally active portion of OX40 ligand covalently linked to a second moiety, e.g., a protein domain. Also included within the definition of OX40L are variants which vary in amino acid sequence from naturally occurring OX4L but which retain the ability to specifically bind to the OX40 receptor. Further included within the definition of OX40L are variants which enhance the biological activity of OX40. OX40 ligand sequences are known in the art and are provided, for example, at GenBank Accession Numbers: NP_003318.

An exemplary human OX40 ligand amino acid sequence is provided below (SEQ ID NO: 19):

MERVQPLEENVGNAARPRFERNKLLLVASVIQGLGLLLCFTYICLHFSAL
QVSHRYPRIQSIKVQFTEYKKEKGFILTSQKEDEIMKVQNNSVIINCDGF
YLISLKGYFSQEVNISLHYQKDEEPLFQLKKVRSVNSLMVASLTYKDKVY
LNVTTDNTSLDDFHVNGGELILIHQNPGEFCVL

By “OX40 agonist” is meant an OX40 ligand that specifically interacts with and increases the biological activity of the OX40 receptor. Desirably, the biological activity is increased by at least about 10%, 20%, 30%, 50%, 70%, 80%, 90%, 95%, or even 100%. In certain aspects, OX40 agonists as disclosed herein include OX40 binding polypeptides, such as anti-OX40 antibodies (e.g., OX40 agonist antibodies), OX40 ligands, or fragments or derivatives of these molecules.

By “OX40 antibody” is meant an antibody that specifically binds OX40. OX40 antibodies include monoclonal and polyclonal antibodies that are specific for OX40 and antigen-binding fragments thereof. In certain aspects, anti-OX40 antibodies as described herein are monoclonal antibodies (or antigen-binding fragments thereof), e.g., murine, humanized, or fully human monoclonal antibodies. In one particular embodiment, the OX40 antibody is an OX40 receptor agonist, such as the mouse anti-human OX40 monoclonal antibody (9B12) described by Weinberg et al., J Immunother 29, 575-585 (2006). In other embodiments, the antibody which specifically binds to OX40, or an antigen-binding fragment thereof binds to the same OX40 epitope as mAb 9B12.

By “OX40 ligand fusion protein (OX40L FP)” is meant a protein that specifically binds the OX40 receptor and increases an immune response. In one embodiment, binding of an OX40 ligand fusion protein to the OX40 receptor enhances a tumor antigen specific immune response by boosting T-cell recognition. Exemplary OX40 ligand fusion proteins are described in U.S. Pat. No. 7,959,925, entitled, “Trimeric OX40 Immunoglobulin Fusion Protein and Methods of Use.” See, for example, U.S. Pat. No. 7,959,925, SEQ ID NO. 8 (SEQ ID NO: 20):

LATDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGKELLGGGSIKQIEDKIEEILS
KIYHIENEIARIKKLIGERGHGGGSNSQVSHRYPRFQSIKVQFTEYKKEK
GFILTSQKEDEIMKVQNNSVIINCDGFYLISLKGYFSQEVNISLHYQKDE
EPLFQLKKVRSVNSLMVASLTYKDKVYLNVTTDNTSLDDFHVNGGELILI
HQNPGEFCVL

Other OX40 ligand fusion proteins are described, for example, in U.S. Pat. No. 6,312,700. In one embodiment, an OX40 ligand fusion protein enhances tumor-specific T-cell immunity. In one embodiment, the OX40 ligand fusion protein is MEDI6383.

By “PD-L1 polypeptide” is meant a polypeptide or fragment thereof having at least about 85% amino acid identity to NCBI Accession No. NP_001254635 and having PD-1 and CD80 binding activity.

By “PD-L1 nucleic acid molecule” is meant a polynucleotide encoding a PD-L1 polypeptide. An exemplary PD-L1 nucleic acid molecule sequence is provided at NCBI Accession No. NM_001267706.

By “anti-PD-L1 antibody” is meant an antibody that selectively binds a PD-L1 polypeptide. Exemplary anti-PD-L1 antibodies are described for example at US20130034559/U.S. Pat. No. 8,779,108 and US20140356353, which is herein incorporated by reference. MEDI4736 is an exemplary anti-PD-L1 antibody. Other anti-PD-L1 antibodies include BMS-936559 (Bristol-Myers Squibb) and MPDL3280A (Roche).

MEDI4736 VL
(SEQ ID NO: 1)
EIVLTQSPOTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIK
MEDI4736 VH
(SEQ ID NO: 2)
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSS
MEDI4736 VH CDR1
(SEQ ID NO: 3)
RYWMS
MEDI4736 VH CDR2
(SEQ ID NO: 4)
NIKQDGSEKYYVDSVKG
MEDI4736 VH CDR3
(SEQ ID NO: 5)
EGGWFGELAFDY
MEDI4736 VL CDR1
(SEQ ID NO: 6)
RASQRVSSSYLA
MEDI4736 VL CDR2
(SEQ ID NO: 7)
DASSRAT
MEDI4736 VL CDR3
(SEQ ID NO: 8)
QQYGSLPWT

By “CTLA-4 polypeptide” is meant a polypeptide having at least 85% amino acid sequence identity to GenBank Accession No. AAL07473.1 or a fragment thereof having T cell inhibitory activity. The sequence of AAL07473.1 is provided below (SEQ ID NO: 21):

gi|5778586|gb|AAL07473.1|AF414120_1 CTLA-4
[Homo sapiens]
MACLFFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
YVKMPPTEPECEKQFQPYFIPIN

By “CTLA-4 nucleic acid molecule” is meant a polynucleotide encoding a CTLA-4 polypeptide. An exemplary CTLA-4 polynucleotide is provided at GenBank Accession No. AAL07473.

By “anti-CTLA-4 antibody” is meant an antibody that selectively binds a CTLA-4 polypeptide. Exemplary anti-CTLA-4 antibodies are described for example at U.S. Pat. Nos. 6,682,736; 7,109,003; 7,123,281; 7,411,057; 7,824,679; 8,143,379; 7,807,797; and 8,491,895 (Tremelimumab is 11.2.1, therein), which are herein incorporated by reference. Tremelimumab is an exemplary anti-CTLA-4 antibody. Tremelimumab sequences are provided below.

Tremelimumab U.S. Pat. No. 6,682,736

Tremelimumab VL
(SEQ ID NO: 9)
PSSLSASVGDRVTITCRASQSINSYLDWYQQKPGKAPKLLIYAASSLQSG
VPSRFSGSGSGTDFTLTISSLQPEDFATYYCQQYYSTPFTFGPGTKVEIK
RTVAAPSVFIFPPSDEQLKSGTASVVCLLNNFYPREAKV
Tremelimumab VH
(SEQ ID NO: 10)
GVVQPGRSLRLSCAASGFTFSSYGMHWVRQAPGKGLEWVAVIWYDGSNKY
YADSVKGRFTISRDNSKNTLYLQMNSLRAEDTAVYYCARDPRGATLYYYY
YGMDVWGQGTTVTVSSASTKGPSVFPLAPCSRSTSESTAALGCLVKDYFP
EPVTVSWNSGALTSGVH
Tremelimumab VH CDR1
(SEQ ID NO: 11)
GFIFSSYGMH
Tremelimumab VH CDR2
(SEQ ID NO: 12)
VIWYDGSNKYYADSV
Tremelimumab VH CDR3
(SEQ ID NO: 13)
DPRGATLYYYYYGMDV
Tremelimumab VL CDR1
(SEQ ID NO: 14)
RASQSINSYLD
Tremelimumab VL CDR2
(SEQ ID NO: 15)
AASSLQS
Tremelimumab VL CDR3
(SEQ ID NO: 16)
QQYYSTPFT

The term “antibody,” as used in this disclosure, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)2, Fv, scFv, Fd, dAb, and other antibody fragments that retain antigen-binding function, i.e., the ability to bind, for example, CTLA-4 or PD-L1, specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)2 fragment in which the two arms of the antibody molecule remain linked and comprise two-antigen binding sites. The F(ab′)2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

The term “mAb” refers to monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “reference” is meant a standard of comparison.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1F show that treatment with a combination of anti-CTLA-4 antibodies, anti-PD-L1 antibodies and OX40 ligand fusion protein was more effective at reducing tumor volumes and increasing survival than monotherapy and dual combination therapies of the agents. FIG. 1A is a graph depicting mean tumor volumes of study groups over time. FIG. 1B shows graphs depicting individual tumor volumes of isotype control (left panel) and untreated subjects (right panel) over time. FIG. 1C shows graph depicting individual tumor volumes of subjects treated with anti-CTLA-4 monoclonal antibody (CTLA-4 mAb; left panel); anti-PD-L1 monoclonal antibody (PD-L1 mAb; center panel); and OX40 ligand fusion protein (mOX40L FP; right panel) over time. FIG. 1D shows graphs depicting individual tumor volumes of subjects treated with anti-CTLA-4 and anti-PD-L1 monoclonal antibodies (CTLA-4 mAb+PD-L1 mAb; top, left panel); anti-PD-L1 monoclonal antibody and OX40 (PD-L1 mAb+mOX40L FP; top, right panel); and anti-CTLA-4 monoclonal antibody and OX40 ligand fusion protein (CTLA-4 mAb+mOX40L FP; bottom, left panel); and a combination of anti-CTLA-4 and anti-PD-L1 monoclonal antibodies and OX40 (CTLA-4 mAb+PD-L1 mAb+mOX40L FP) over time. FIG. 1E is a graph showing percent survival in untreated, isotype control, CTLA-4 mAb, PD-L1 mAb, and mOX40L FP study groups over time. FIG. 1F is a graph showing percent survival in CTLA-4 mAb+PD-L1 mAb, isotype control, CTLA-4 mAb+mOX40L FP, PD-L1 mAb+mOX40L FP, and CTLA-4 mAb+PD-L1 mAb+mOX40L FP FP study groups over time. Eleven C57BL/6 mice in each group were inoculated subcutaneously (SC) on Day 1 with MCA205 cells. Control article (isotype control) and the test articles anti-CTLA-4 mAb and anti-PD-L1 mAb were administered intraperitoneally (IP) on Days 11, 15, 18 and 22; test article mOX40L FP was administered IP on Days 11 and 15. A comparison between test article-treated and the isotype control-treated animals was made, and intergroup differences were analyzed for statistical significance by the method described in Section 6.6 using GraphPad Prism 6.0 software. Error bars represent standard error of the mean. IP=intraperitoneal; SC=subcutaneous; TGI=tumor growth inhibition.

### DETAILED DESCRIPTION OF THE INVENTION

The present invention features compositions and methods that are useful for treating cancer, comprising a combination of an anti-CTLA-4 antibody, an anti-PD-L1 antibody, and OX40 agonist (e.g., OX40 ligand fusion protein). As reported herein below, treatment with these agents reduced tumor volume and increased survival in a mouse tumor model.

Provided herein are methods for treating solid tumors. The methods provided include administering an effective amount of MEDI4736 or an antigen-binding fragment thereof, tremelimumab or an antigen-binding fragment thereof, and OX40 ligand fusion protein or active fragment thereof. In various embodiments the solid tumor is, but is not limited to, ovarian cancer, breast cancer (e.g., triple negative breast cancer), colorectal cancer, prostate cancer, cervical cancer, uterine cancer, testicular cancer, bladder cancer, head and neck cancer, melanoma, pancreatic cancer, renal cell carcinoma, and lung cancer (e.g., non-small cell lung cancer (NSCLC)). There are three main subtypes of NSCLC: squamous cell carcinoma, adenocarcinoma, and large cell (undifferentiated) carcinoma. Other subtypes include adenosquamous carcinoma and sarcomatoid carcinoma.

Anti-Tumor Therapy

Provided herein are methods for treating cancer, comprising administration of anti-CTLA4 antibody, anti-PD-L1 antibody, and OX40 agonist (e.g., an OX40 ligand fusion protein, OX40 agonist antibody). Administration of an anti-CTLA4 antibody, anti-PD-L1 antibody, and OX40 ligand fusion protein resulted in a reduction in tumor volume and increased survival in a mouse tumor model. In certain aspects, a patient presenting with a solid tumor is administered an anti-CTLA4 antibody (e.g., tremelimumab), an anti-PD-L1 (MEDI4736), and an OX40 ligand fusion protein (e.g., MEDI6383). In certain aspects, administration of an anti-CTLA4 antibody (e.g., tremelimumab), an anti-PD-L1 (MEDI4736), and an OX40 ligand fusion protein (e.g., MEDI6383) according to the methods provided herein is through parenteral administration (e.g., intravenous infusion or subcutaneous injection). In certain aspects, the anti-CTLA4 antibody (e.g., tremelimumab), anti-PD-L1 (MEDI4736), and OX40 ligand fusion protein (e.g., MEDI6383) are administered as a single pharmaceutical composition.

Effective treatment with a cancer therapy including an anti-CTLA4 antibody, anti-PD-L1 antibody, and OX40 agonist includes, for example, reducing the rate of progression of the cancer, retardation or stabilization of tumor or metastatic growth, tumor shrinkage, and/or tumor regression, either at the site of a primary tumor, or in one or more metastases. In some aspects the reduction or retardation of tumor growth can be statistically significant. A reduction in tumor growth can be measured by comparison to the growth of patient's tumor at baseline, against an expected tumor growth, against an expected tumor growth based on a large patient population, or against the tumor growth of a control population. In other embodiments, the methods of the invention increase survival.

Clinical response to administration of a cancer therapy including an anti-CTLA4 antibody, anti-PD-L1 antibody, and OX40 ligand fusion protein can be assessed using diagnostic techniques known to clinicians, including but not limited to magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, and chromatography.

T Cell Modulatory Pathways

There is mounting evidence that T cells control tumor growth and survival in cancer patients, both in early and late stages of the disease. However, tumor-specific T-cell responses are difficult to mount and sustain in cancer patients.

T cell modulatory pathways receiving significant attention signal through cytotoxic T lymphocyte antigen-4 (CTLA-4, CD152), programmed death ligand 1 (PD-L1, also known as B7H-1 or CD274) and OX40 (CD134; TNFRSF4).

CTLA-4 is expressed on activated T cells and serves as a co-inhibitor to keep T cell responses in check following CD28-mediated T cell activation. CTLA-4 is believed to regulate the amplitude of the early activation of naïve and memory T cells following TCR engagement and to be part of a central inhibitory pathway that affects both antitumor immunity and autoimmunity. CTLA-4 is expressed on T cells, and the expression of its ligands CD80 (B7.1) and CD86 (B7.2), is largely restricted to antigen-presenting cells, T cells, and other immune mediating cells. Antagonistic anti-CTLA-4 antibodies that block the CTLA-4 signaling pathway have been reported to enhance T cell activation. One such antibody, ipilimumab, was approved by the FDA in 2011 for the treatment of metastatic melanoma. Another anti-CTLA-4 antibody, tremelimumab, was tested in phase III trials for the treatment of advanced melanoma but did not significantly increase the overall survival of patients compared to the standard of care (temozolomide or dacarbazine) at that time.

PD-L1 is also part of a complex system of receptors and ligands that are involved in controlling T cell activation. In normal tissue, PD-L1 is expressed on T cells, B cells, dendritic cells, macrophages, mesenchymal stem cells, bone marrow-derived mast cells, as well as various nonhematopoietic cells. Its normal function is to regulate the balance between T-cell activation and tolerance through interaction with its two receptors: programmed death 1 (also known as PD-1 or CD279) and CD80 (also known as B7-1 or B7.1). PD-L1 is also expressed by tumors and acts at multiple sites to help tumors evade detection and elimination by the host immune system. PD-L1 is expressed in a broad range of cancers with a high frequency. In some cancers, expression of PD-L1 has been associated with reduced survival and unfavorable prognosis. Antibodies that block the interaction between PD-L1 and its receptors (e.g., PD-1) are able to relieve PD-L1-dependent immunosuppressive effects and enhance the cytotoxic activity of antitumor T cells in vitro.

OX40 (CD134; TNFRSF4) is a tumor necrosis factor receptor (TNFR) found primarily on activated CD4+ and CD8+ T cells, regulatory T cells (Treg) and natural killer (NK) cells (Croft et al, 2009). OX40 has one known endogenous ligand, OX40 ligand (OX40L; CD152; TNFSF4), which exists in a trimeric form and can cluster OX40, resulting in potent cell signaling events within T cells (Croft et al, 2009). Signaling through OX40 on activated CD4+ and CD8+ T cells leads to enhanced cytokine production, granzyme and perforin release, and expansion of effector and memory T cell pools (Jensen et al, 2010). In addition, OX40 signaling on Treg cells inhibits expansion of Tregs, shuts down the induction of Tregs, and blocks Treg-suppressive function (Voo et al, 2013; Vu et al, 2007).

Immunohistochemistry studies and early flow cytometry analyses showed that OX40 is expressed on T cells infiltrating a broad range of human cancers (Baruah et al, 2011; Curti et al, 2013; Ladanyi et al, 2004; Petty et al, 2002; Ramstad et al, 2000; Sarff et al, 2008; Vetto et al, 1997). OX40 expression on tumor-infiltrating lymphocytes correlates with longer survival in several human cancers, suggesting that OX40 signals may play an important role in establishing an anti-tumor immune response (Ladanyi et al, 2004; Petty et al, 2002).

In a variety of nonclinical mouse tumor models, agonists of OX40, including antibodies and OX40 ligand fusion proteins, have been used successfully with promising results (Kjaergaard et al, 2000; Ndhlovu et al, 2001; Weinberg et al, 2000). Co-stimulating T cells through OX40 agonists promoted anti-tumor activity that in some cases was durable, providing long-lasting protection against subsequent tumor challenge (Weinberg et al, 2000). Treg-cell inhibition and co-stimulation of effector T cells were shown to be necessary for tumor growth inhibition by OX40 agonists (Piconese et al, 2008).

Anti-PD-L1 Antibodies

MEDI4736 is an exemplary anti-PD-L1 antibody that is selective for PD-L1 and blocks the binding of PD-L1 to the PD-1 and CD80 receptors. MEDI4736 can relieve PD-L1-mediated suppression of human T-cell activation in vitro and inhibits tumor growth in a xenograft model via a T-cell dependent mechanism.

Information regarding MEDI4736 (or fragments thereof) for use in the methods provided herein can be found in U.S. Pat. No. 8,779,108, the disclosure of which is incorporated herein by reference in its entirety. The fragment crystallizable (Fc) domain of MEDI4736 contains a triple mutation in the constant domain of the IgG1 heavy chain that reduces binding to the complement component C1q and the Fcγ receptors responsible for mediating antibody-dependent cell-mediated cytotoxicity (ADCC).

MEDI4736 and antigen-binding fragments thereof for use in the methods provided herein comprises a heavy chain and a light chain or a heavy chain variable region and a light chain variable region. In a specific aspect, MEDI4736 or an antigen-binding fragment thereof for use in the methods provided herein comprises a light chain variable region and a heavy chain variable region. In a specific aspect, MEDI4736 or an antigen-binding fragment thereof for use in the methods provided herein comprises a heavy chain variable region and a light chain variable region, wherein the heavy chain variable region comprises the Kabat-defined CDR1, CDR2, and CDR3 sequences shown herein above, and wherein the light chain variable region comprises the Kabat-defined CDR1, CDR2, and CDR3 sequences shown herein above. Those of ordinary skill in the art would easily be able to identify Chothia-defined, Abm-defined or other CDR definitions known to those of ordinary skill in the art. In a specific aspect, MEDI4736 or an antigen-binding fragment thereof for use in the methods provided herein comprises the variable heavy chain and variable light chain CDR sequences of the 2.14H9OPT antibody as disclosed in U.S. Pat. No. 8,779,108, which is herein incorporated by reference in its entirety.

Anti-CTLA-4 Antibodies

Antibodies that specifically bind CTLA-4 and inhibit CTLA-4 activity are useful for enhancing an anti-tumor immune response. Information regarding tremelimumab (or antigen-binding fragments thereof) for use in the methods provided herein can be found in U.S. Pat. No. 6,682,736 (where it is referred to as 11.2.1), the disclosure of which is incorporated herein by reference in its entirety. Tremelimumab (also known as CP-675,206, CP-675, CP-675206, and ticilimumab) is a human IgG2 monoclonal antibody that is highly selective for CTLA-4 and blocks binding of CTLA-4 to CD80 (B7.1) and CD86 (B7.2). It has been shown to result in immune activation in vitro and some patients treated with tremelimumab have shown tumor regression.

Tremelimumab for use in the methods provided herein comprises a heavy chain and a light chain or a heavy chain variable region and a light chain variable region. In a specific aspect, tremelimumab or an antigen-binding fragment thereof for use in the methods provided herein comprises a light chain variable region comprising the amino acid sequences shown herein above and a heavy chain variable region comprising the amino acid sequence shown herein above. In a specific aspect, tremelimumab or an antigen-binding fragment thereof for use in the methods provided herein comprises a heavy chain variable region and a light chain variable region, wherein the heavy chain variable region comprises the Kabat-defined CDR1, CDR2, and CDR3 sequences shown herein above, and wherein the light chain variable region comprises the Kabat-defined CDR1, CDR2, and CDR3 sequences shown herein above. Those of ordinary skill in the art would easily be able to identify Chothia-defined, Abm-defined or other CDR definitions known to those of ordinary skill in the art. In a specific aspect, tremelimumab or an antigen-binding fragment thereof for use in the methods provided herein comprises the variable heavy chain and variable light chain CDR sequences of the 11.2.1 antibody as disclosed in U.S. Pat. No. 6,682,736, which is herein incorporated by reference in its entirety.

Other anti-CTLA-4 antibodies are described, for example, in US 20070243184. In one embodiment, the anti-CTLA-4 antibody is Ipilimumab, also termed MDX-010; BMS-734016.

OX40 Agonists

OX40 agonists interact with the OX40 receptor on CD4⁺ T-cells during, or shortly after, priming by an antigen resulting in an increased response of the CD4⁺ T-cells to the antigen. An OX40 agonist interacting with the OX40 receptor on antigen specific CD4⁺ T-cells can increase T cell proliferation as compared to the response to antigen alone. The elevated response to the antigen can be maintained for a period of time substantially longer than in the absence of an OX40 agonist. Thus, stimulation via an OX40 agonist enhances the antigen specific immune response by boosting T-cell recognition of antigens, e.g., tumor cells. OX40 agonists are described, for example, in U.S. Pat. Nos. 6,312,700, 7,504,101, 7,622,444, and 7,959,925, which are incorporated herein by reference in their entireties. Methods of using such agonists in cancer treatment are described, for example, in WO/2013/119202 and in WO/2013/130102, each of which are incorporated herein by reference in its entirety.

OX40 agonists include, but are not limited to OX40 binding molecules, e.g., binding polypeptides, e.g., OX40 ligand (“OX40L”) or an OX40-binding fragment, variant, or derivative thereof, such as soluble extracellular ligand domains and OX40L fusion proteins, and anti-OX40 antibodies (for example, monoclonal antibodies such as humanized monoclonal antibodies), or an antigen-binding fragment, variant or derivative thereof. Examples of anti-OX40 monoclonal antibodies are described, for example, in U.S. Pat. Nos. 5,821,332 and 6,156,878, the disclosures of which are incorporated herein by reference in their entireties. In certain embodiments, the anti-OX40 monoclonal antibody is 9B12, or an antigen-binding fragment, variant, or derivative thereof, as described in Weinberg, A. D., et al. J Immunother 29, 575-585 (2006), which is incorporated herein by reference in its entirety.

In certain aspects this disclosure provides a humanized anti-OX40 antibody or an antigen-binding fragment thereof comprising an antibody VH and an antibody VL, wherein the VL comprises an amino acid sequence at least 70%, 75%, 80%, 85%, 90%, 95%, or 100% identical to the reference amino acid sequence

(SEQ ID NO: 22)
DIQMTQSPSSLSASVGDRVTITCRASQDISNYLNWYQQKPGKAPKLLIYY
TSKLHSGVPSRFSGSGSGTDYTLTISSLQPEDFATYYCQQGSALPWTFGQ
GTKVEIK
or
(SEQ ID NO: 23)
DIQMTQSPSSLSASVGDRVTITCRASQDISNYLNWYQQKPGKAVKLLIYY
TSKLHSGVPSRFSGSGSRTDYTLTISSLQPEDFATYYCQQGSALPWTFGQ
GTKVEIK

In one aspect, the disclosure provides a humanized anti-OX40 antibody or an antigen-binding fragment thereof comprising an antibody VH and an antibody VL, where the VL comprises the amino acid sequence

(SEQ ID NO: 22)
DIQMTQSPSSLSASVGDRVTITCRASQDISNYLNWYQQKPGKAPKLLIYY
TSKLHSGVPSRFSGSGSGTDYTLTISSLQPEDFATYYCQQGSALPWTFGQ
GTKVEIK

and the VH comprises the amino acid sequence

(SEQ ID NO: 24)
QVQLQESGPGLVKPSQTLSLTCAVYGGSFSSGYWNWIRKHPGKGLEYIGY
ISYNGITYHNPSLKSRITINRDTSKNQYSLQLNSVTPEDTAVYYCARYKY
DYDGGHAMDYWGQGTLVTVSS.

In certain aspects the disclosure provides a humanized anti-OX40 antibody or an antigen-binding fragment thereof comprising an antibody heavy chain or fragment thereof and an antibody light chain or fragment thereof, where the heavy chain comprises the amino acid sequence QVQLQESGPGLVKPSQTLSLTCAVYGGSFSSGYWNWIRKHPGKGLEYIGYISYNGITYH NPSLKSRITINRDTSKNQYSLQLNSVTPEDTAVYYCARYKYDYDGGHAMDYWGQGTLV TVSSASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKRVEPKSCDKTHTCPPCPAPE LLGGPSVFLFPPKPKDTLMISRTPEVTCVVVDVSHEDPEVKFNWYVDGVEVHNAKTKPR EEQYNSTYRVVSVLTVLHQDWLNGKEYKCKVSNKALPAPIEKTISKAKGQPREPQVYTL PPSREEMTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO: 25), and the light chain comprises the amino acid sequence

(SEQ ID NO: 26)
DIQMTQSPSSLSASVGDRVTITCRASQDISNYLNWYQQKPGKAPKLLIYY
TSKLHSGVPSRFSGSGSGTDYTLTISSLQPEDFATYYCQQGSALPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In other embodiments, the antibody which specifically binds to OX40, or an antigen-binding fragment thereof binds to the same OX40 epitope as mAb 9B12.

An exemplary humanized OX40 antibody is described by Morris et al., Mol Immunol. May 2007; 44(12): 3112-3121, and has the following sequence (SEQ ID NO: 27):

APLATDKTHTCPPCPAPELLGGPSVFLFPPKPKDTLMISRTPEV
TCVVVDVSHEDPEVKFNWYVDGVEVHNAKTKPREEQYNSTYRVV
SVLTVLHQDWLNGKEYKCKVSNKALPAPIEKTISKAKGQPREPQ
VYTLPPSREEMTKNQVSLTCLVKGFYPSDIAVEWESNGQPENNY
KTTPPVLDSDGSFFLYSKLTVDKSRWQQGNVFSCSVMHEALHNH
YTQKSLSLSPGKELLGGGSIKQIEDKIEEILSKIYHIENEIARI
KKLIGERGHGGGSNSQVSHRYPRFQSIKVQFTEYKKEKGFILTS
QKEDEIMKVQNNSVIINCDGFYLISLKGYFSQEVNISLHYQKDE
EPLFQLKKVRSVNSLMVASLTYKDKVYLNVTTDNTSLDDFHVNG
GELILIHQNPGEFCVL

9B12 is a murine IgG1, anti-OX40 mAb directed against the extracellular domain of human OX40 (CD134) (Weinberg, A. D., et al. J Immunother 29, 575-585 (2006)). It was selected from a panel of anti-OX40 monoclonal antibodies because of its ability to elicit an agonist response for OX40 signaling, stability, and for its high level of production by the hybridoma. For use in clinical applications, 9B12 mAb is equilibrated with phosphate buffered saline, pH 7.0, and its concentration is adjusted to 5.0 mg/ml by diafiltration.

“OX40 ligand” (“OX40L”) (also variously termed tumor necrosis factor ligand superfamily member 4, gp34, TAX transcriptionally-activated glycoprotein-1, and CD252) is found largely on antigen presenting cells (APCs), and can be induced on activated B cells, dendritic cells (DCs), Langerhans cells, plamacytoid DCs, and macrophages (Croft, M., (2010) Ann Rev Immunol 28:57-78). Other cells, including activated T cells, NK cells, mast cells, endothelial cells, and smooth muscle cells can express OX40L in response to inflammatory cytokines (Id.). OX40L specifically binds to the OX40 receptor. The human protein is described in U.S. Pat. No. 6,156,878. The mouse OX40L is described in U.S. Pat. No. 5,457,035. OX40L is expressed on the surface of cells and includes an intracellular, a transmembrane and an extracellular receptor-binding domain. A functionally active soluble form of OX40L can be produced by deleting the intracellular and transmembrane domains as described, e.g., in U.S. Pat. Nos. 5,457,035; 6,312,700; 6,156,878; 6,242,566; 6,528,055; 6,528,623; 7,098,184; and 7,125,670, the disclosures of which are incorporated herein for all purposes. A functionally active form of OX40L is a form that retains the capacity to bind specifically to OX40, that is, that possesses an OX40 “receptor binding domain.” An example is amino acids 51 to 183 of human OX40L. Methods of determining the ability of an OX40L molecule or derivative to bind specifically to OX40 are discussed below. Methods of making and using OX40L and its derivatives (such as derivatives that include an OX40 binding domain) are described in U.S. Pat. Nos. 6,156,878; 6,242,566; 6,528,055; 6,528,623; 7,098,184; and 7,125,670, which also describe proteins comprising the soluble form of OX40L linked to other peptides, such as human immunoglobulin (“Ig”) Fc regions, that can be produced to facilitate purification of OX40 ligand from cultured cells, or to enhance the stability of the molecule after in vivo administration to a mammal (see also, U.S. Pat. Nos. 5,457,035 and 7,959,925, both of which are incorporated by reference herein in their entireties).

As used herein, the term “OX40L” includes the entire OX40 ligand, soluble OX40 ligand, and functionally active portions of the OX40 ligand. Also included within the definition of OX40L are OX40 ligand variants which vary in amino acid sequence from naturally occurring OX40 ligand molecules but which retain the ability to specifically bind to an OX40 receptor. Such variants are described in U.S. Pat. Nos. 5,457,035; 6,156,878; 6,242,566; 6,528,055; 6,528,623; 7,098,184; and 7,125,670. In a related embodiment, the disclosure provides mutants of OX40L which have lost the ability to specifically bind to OX40, for example amino acids 51 to 183, in which the phenylalanine at position 180 of the receptor-binding domain of human OX40L has been replaced with alanine (F180A).

OX40 agonists include a fusion protein in which one or more domains of OX40L is covalently linked to one or more additional protein domains. Exemplary OX40L fusion proteins that can be used as OX40 agonists are described in U.S. Pat. No. 6,312,700, the disclosure of which is incorporated herein by reference in its entirety. In one embodiment, an OX40 agonist includes an OX40L fusion polypeptide that self-assembles into a multimeric (e.g., trimeric or hexameric) OX40L fusion protein. Such fusion proteins are described, e.g., in U.S. Pat. No. 7,959,925, which is incorporated by reference herein in its entirety. The multimeric OX40L fusion protein exhibits increased efficacy in enhancing antigen specific immune response in a subject, particularly a human subject, due to its ability to spontaneously assemble into highly stable trimers and hexamers.

In another embodiment, an OX40 agonist capable of assembling into a multimeric form includes a fusion polypeptide comprising in an N-terminal to C-terminal direction: an immunoglobulin domain, wherein the immunoglobulin domain includes an Fc domain, a trimerization domain, wherein the trimerization domain includes a coiled coil trimerization domain, and a receptor binding domain, wherein the receptor binding domain is an OX40 receptor binding domain, e.g., an OX40L or an OX40-binding fragment, variant, or derivative thereof, where the fusion polypeptide can self-assemble into a trimeric fusion protein. In one aspect, an OX40 agonist capable of assembling into a multimeric form is capable of binding to the OX40 receptor and stimulating at least one OX40 mediated activity. In certain aspects, the OX40 agonist includes an extracellular domain of OX40 ligand.

The trimerization domain of an OX40 agonist capable of assembling into a multimeric form serves to promote self-assembly of individual OX40L fusion polypeptide molecules into a trimeric protein. Thus, an OX40L fusion polypeptide with a trimerization domain self-assembles into a trimeric OX40L fusion protein. In one aspect, the trimerization domain is an isoleucine zipper domain or other coiled coil polypeptide structure. Exemplary coiled coil trimerization domains include: TRAF2 (GENBANK® Accession No. Q12933, amino acids 299-348; Thrombospondin 1 (Accession No. PO7996, amino acids 291-314; Matrilin-4 (Accession No. O95460, amino acids 594-618; CMP (matrilin-1) (Accession No. NP-002370, amino acids 463-496; HSF1 (Accession No. AAX42211, amino acids 165-191; and Cubilin (Accession No. NP-001072, amino acids 104-138. In certain specific aspects, the trimerization domain includes a TRAF2 trimerization domain, a Matrilin-4 trimerization domain, or a combination thereof.

MEDI6383 is a human OX40 ligand IgG4P fusion protein that specifically binds to, and triggers signaling by, the human OX40 receptor, a member of the TNFR superfamily. MEDI6383 is composed of three distinct domains: (1) human OX40 ligand extracellular receptor binding domains (RBDs) that form homotrimers and bind the OX40 receptor; (2) isoleucine zipper trimerization domains derived from TNFR-associated factor 2 that stabilize the homotrimeric structure of the OX40 ligand RBDs; and (3) human IgG4 fragment crystallizable gamma (Fcγ) domains that facilitate Fcγ receptor clustering of the fusion protein when bound to OX40 receptors, and contain a serine to proline substitution in the hinge regions (IgG4P) to promote stability of two sets of OX40 ligand RBD homotrimers.

In particular embodiments, an OX40 agonist is modified to increase its serum half-life. For example, the serum half-life of an OX40 agonist can be increased by conjugation to a heterologous molecule such as serum albumin, an antibody Fc region, or PEG. In certain embodiments, OX40 agonists can be conjugated to other therapeutic agents or toxins to form immunoconjugates and/or fusion proteins. In certain aspects, an OX40 agonist can be formulated so as to facilitate administration and promote stability of the active agent.

Antibodies

Antibodies that selectively bind CTLA-4 and PD-L1, and inhibit the binding or activation of CTLA-4 and PD-L1 are useful in the methods of the invention. Antibodies that selectively bind and activate OX40 are useful in the methods of the invention.

In general, antibodies can be made, for example, using traditional hybridoma techniques (Kohler and Milstein (1975) Nature, 256: 495-499), recombinant DNA methods (U.S. Pat. No. 4,816,567), or phage display performed with antibody, libraries (Clackson et al. (1991) Nature, 352: 624-628; Marks et al. (1991) J. Mol. Biol., 222: 581-597). For other antibody production techniques, see also Antibodies: A Laboratory Manual, eds. Harlow et al., Cold Spring Harbor Laboratory, 1988. The invention is not limited to any particular source, species of origin, method of production.

Intact antibodies, also known as immunoglobulins, are typically tetrameric glycosylated proteins composed of two light (L) chains of approximately 25 kDa each and two heavy (H) chains of approximately 50 kDa each. Two types of light chain, designated as the λ chain and the κ chain, are found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2.

The subunit structures and three-dimensional configurations of different classes of immunoglobulins are well known in the art. For a review of antibody structure, see Harlow et al., supra. Briefly, each light chain is composed of an N-terminal variable domain (VL) and a constant domain (CL). Each heavy chain is composed of an N-terminal variable domain (VH), three or four constant domains (CH), and a hinge region. The CH domain most proximal to VH is designated as CH1. The VH and VL domains consist of four regions of relatively conserved sequence called framework regions (FR1, FR2, FR3, and FR4), which form a scaffold for three regions of hypervariable sequence called complementarity determining regions (CDRs). The CDRs contain most of the residues responsible for specific interactions with the antigen. The three CDRs are referred to as CDR1, CDR2, and CDR3. CDR constituents on the heavy chain are referred to as H1, H2, and H3, while CDR constituents on the light chain are referred to as L1, L2, and L3, accordingly. CDR3 and, particularly H3, are the greatest source of molecular diversity within the antigen-binding domain. H3, for example, can be as short as two amino acid residues or greater than 26.

The Fab fragment (Fragment antigen-binding) consists of the VH-CH1 and VL-CL domains covalently linked by a disulfide bond between the constant regions. To overcome the tendency of non-covalently linked VH and VL domains in the Fv to dissociate when co-expressed in a host cell, a so-called single chain (sc) Fv fragment (scFv) can be constructed. In a scFv, a flexible and adequately long polypeptide links either the C-terminus of the VH to the N-terminus of the VL or the C-terminus of the VL to the N-terminus of the VH. Most commonly, a 15-residue (Gly4Ser)3 peptide (SEQ ID NO: 28) is used as a linker but other linkers are also known in the art.

Antibody diversity is a result of combinatorial assembly of multiple germline genes encoding variable regions and a variety of somatic events. The somatic events include recombination of variable gene segments with diversity (D) and joining (J) gene segments to make a complete VH region and the recombination of variable and joining gene segments to make a complete VL region. The recombination process itself is imprecise, resulting in the loss or addition of amino acids at the V(D)J junctions. These mechanisms of diversity occur in the developing B cell prior to antigen exposure. After antigenic stimulation, the expressed antibody genes in B cells undergo somatic mutation.

Based on the estimated number of germline gene segments, the random recombination of these segments, and random VH-VL pairing, up to 1.6×10⁷ different antibodies could be produced (Fundamental Immunology, 3rd ed., ed. Paul, Raven Press, New York, N.Y., 1993). When other processes which contribute to antibody diversity (such as somatic mutation) are taken into account, it is thought that upwards of 1×10¹⁰ different antibodies could be potentially generated (Immunoglobulin Genes, 2nd ed., eds. Jonio et al., Academic Press, San Diego, Calif., 1995). Because of the many processes involved in antibody diversity, it is highly unlikely that independently generated antibodies will have identical or even substantially similar amino acid sequences in the CDRs.

The sequences of exemplary anti-CTLA-4 and anti-PD-L1 CDRs are provided herein. The structure for carrying a CDR will generally be an antibody heavy or light chain or a portion thereof, in which the CDR is located at a location corresponding to the CDR of naturally occurring VH and VL. The structures and locations of immunoglobulin variable domains may be determined, for example, as described in Kabat et al., Sequences of Proteins of Immunological Interest, No. 91-3242, National Institutes of Health Publications, Bethesda, Md., 1991.

Antibodies of the invention (e.g., anti-CTLA-4, anti-PD-L1, anti-OX40) may optionally comprise antibody constant regions or parts thereof. For example, a VL domain may have attached, at its C terminus, antibody light chain constant domains including human Cκ or Cλ chains. Similarly, a specific antigen-binding domain based on a VH domain may have attached all or part of an immunoglobulin heavy chain derived from any antibody isotope, e.g., IgG, IgA, IgE, and IgM and any of the isotope sub-classes, which include but are not limited to, IgG1 and IgG4.

One of ordinary skill in the art will recognize that the antibodies of this invention may be used to detect, measure, and inhibit proteins that differ somewhat from CTLA-4 and PD-L1. The antibodies are expected to retain the specificity of binding so long as the target protein comprises a sequence which is at least about 60%, 70%, 80%, 90%, 95%, or more identical to any sequence of at least 100, 80, 60, 40, or 20 of contiguous amino acids described herein. The percent identity is determined by standard alignment algorithms such as, for example, Basic Local Alignment Tool (BLAST) described in Altshul et al. (1990) J. Mol. Biol., 215: 403-410, the algorithm of Needleman et al. (1970) J. Mol. Biol., 48: 444-453, or the algorithm of Meyers et al. (1988) Comput. Appl. Biosci., 4: 11-17.

In addition to the sequence homology analyses, epitope mapping (see, e.g., Epitope Mapping Protocols, ed. Morris, Humana Press, 1996) and secondary and tertiary structure analyses can be carried out to identify specific 3D structures assumed by the disclosed antibodies and their complexes with antigens. Such methods include, but are not limited to, X-ray crystallography (Engstom (1974) Biochem. Exp. Biol., 11:7-13) and computer modeling of virtual representations of the presently disclosed antibodies (Fletterick et al. (1986) Computer Graphics and Molecular Modeling, in Current Communications in Molecular Biology, Cold Spring Harbor Laboratory, Cold Spring Harbor, N.Y.).

Derivatives

Antibodies of the invention (e.g., anti-CTLA-4, anti-PD-L1, anti-OX40) may include variants of these sequences that retain the ability to specifically bind their targets. Such variants may be derived from the sequence of these antibodies by a skilled artisan using techniques well known in the art. For example, amino acid substitutions, deletions, or additions, can be made in the FRs and/or in the CDRs. While changes in the FRs are usually designed to improve stability and immunogenicity of the antibody, changes in the CDRs are typically designed to increase affinity of the antibody for its target. Variants of FRs also include naturally occurring immunoglobulin allotypes. Such affinity-increasing changes may be determined empirically by routine techniques that involve altering the CDR and testing the affinity antibody for its target. For example, conservative amino acid substitutions can be made within any one of the disclosed CDRs. Various alterations can be made according to the methods described in Antibody Engineering, 2nd ed., Oxford University Press, ed. Borrebaeck, 1995. These include but are not limited to nucleotide sequences that are altered by the substitution of different codons that encode a functionally equivalent amino acid residue within the sequence, thus producing a “silent” change. For example, the nonpolar amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid.

Derivatives and analogs of antibodies of the invention can be produced by various techniques well known in the art, including recombinant and synthetic methods (Maniatis (1990) Molecular Cloning, A Laboratory Manual, 2nd ed., Cold Spring Harbor Laboratory, Cold Spring Harbor, N.Y., and Bodansky et al. (1995) The Practice of Peptide Synthesis, 2nd ed., Spring Verlag, Berlin, Germany).

In one embodiment, a method for making a VH domain which is an amino acid sequence variant of a VH domain of the invention comprises a step of adding, deleting, substituting, or inserting one or more amino acids in the amino acid sequence of the presently disclosed VH domain, optionally combining the VH domain thus provided with one or more VL domains, and testing the VH domain or VH/VL combination or combinations for specific binding to the antigen. An analogous method can be employed in which one or more sequence variants of a VL domain disclosed herein are combined with one or more VH domains.

Analogous shuffling or combinatorial techniques are also disclosed by Stemmer (Nature (1994) 370: 389-391), who describes the technique in relation to a β-lactamase gene but observes that the approach may be used for the generation of antibodies.

In further embodiments, one may generate novel VH or VL regions carrying one or more sequences derived from the sequences disclosed herein using random mutagenesis of one or more selected VH and/or VL genes. One such technique, error-prone PCR, is described by Gram et al. (Proc. Nat. Acad. Sci. U.S.A. (1992) 89: 3576-3580).

Another method that may be used is to direct mutagenesis to CDRs of VH or VL genes. Such techniques are disclosed by Barbas et al. (Proc. Nat. Acad. Sci. U.S.A. (1994) 91: 3809-3813) and Schier et al. (J. Mol. Biol. (1996) 263: 551-567).

Similarly, one or more, or all three CDRs may be grafted into a repertoire of VH or VL domains, which are then screened for an antigen-binding fragment specific for CTLA-4 or PD-L1.

A portion of an immunoglobulin variable domain will comprise at least one of the CDRs substantially as set out herein and, optionally, intervening framework regions from the scFv fragments as set out herein. The portion may include at least about 50% of either or both of FR1 and FR4, the 50% being the C-terminal 50% of FR1 and the N-terminal 50% of FR4. Additional residues at the N-terminal or C-terminal end of the substantial part of the variable domain may be those not normally associated with naturally occurring variable domain regions. For example, construction of antibodies by recombinant DNA techniques may result in the introduction of N- or C-terminal residues encoded by linkers introduced to facilitate cloning or other manipulation steps. Other manipulation steps include the introduction of linkers to join variable domains to further protein sequences including immunoglobulin heavy chain constant regions, other variable domains (for example, in the production of diabodies), or proteinaceous labels as discussed in further detail below.

A skilled artisan will recognize that antibodies of the invention may comprise antigen-binding fragments containing only a single CDR from either VL or VH domain. Either one of the single chain specific binding domains can be used to screen for complementary domains capable of forming a two-domain specific antigen-binding fragment capable of, for example, binding to CTLA-4 and PD-L1.

Antibodies of the invention (e.g., anti-CTLA-4 and/or anti-PD-L1) described herein can be linked to another functional molecule, e.g., another peptide or protein (albumin, another antibody, etc.). For example, the antibodies can be linked by chemical cross-linking or by recombinant methods. The antibodies may also be linked to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192; or 4,179,337. The antibodies can be chemically modified by covalent conjugation to a polymer, for example, to increase their circulating half-life. Exemplary polymers and methods to attach them are also shown in U.S. Pat. Nos. 4,766,106; 4,179,337; 4,495,285, and 4,609,546.

The disclosed antibodies may also be altered to have a glycosylation pattern that differs from the native pattern. For example, one or more carbohydrate moieties can be deleted and/or one or more glycosylation sites added to the original antibody. Addition of glycosylation sites to the presently disclosed antibodies may be accomplished by altering the amino acid sequence to contain glycosylation site consensus sequences known in the art. Another means of increasing the number of carbohydrate moieties on the antibodies is by chemical or enzymatic coupling of glycosides to the amino acid residues of the antibody. Such methods are described in WO 87/05330, and in Aplin et al. (1981) CRC Crit. Rev. Biochem., 22: 259-306. Removal of any carbohydrate moieties from the antibodies may be accomplished chemically or enzymatically, for example, as described by Hakimuddin et al. (1987) Arch. Biochem. Biophys., 259: 52; and Edge et al. (1981) Anal. Biochem., 118: 131 and by Thotakura et al. (1987) Meth. Enzymol., 138: 350. The antibodies may also be tagged with a detectable, or functional, label. Detectable labels include radiolabels such as 1311 or 99Tc, which may also be attached to antibodies using conventional chemistry. Detectable labels also include enzyme labels such as horseradish peroxidase or alkaline phosphatase. Detectable labels further include chemical moieties such as biotin, which may be detected via binding to a specific cognate detectable moiety, e.g., labeled avidin.

Antibodies, in which CDR sequences differ only insubstantially from those set forth herein are encompassed within the scope of this invention. Typically, an amino acid is substituted by a related amino acid having similar charge, hydrophobic, or stereochemical characteristics. Such substitutions would be within the ordinary skills of an artisan. Unlike in CDRs, more substantial changes can be made in FRs without adversely affecting the binding properties of an antibody. Changes to FRs include, but are not limited to, humanizing a non-human derived or engineering certain framework residues that are important for antigen contact or for stabilizing the binding site, e.g., changing the class or subclass of the constant region, changing specific amino acid residues which might alter the effector function such as Fc receptor binding, e.g., as described in U.S. Pat. Nos. 5,624,821 and 5,648,260 and Lund et al. (1991) J. Immun. 147: 2657-2662 and Morgan et al. (1995) Immunology 86: 319-324, or changing the species from which the constant region is derived.

One of skill in the art will appreciate that the modifications described above are not all-exhaustive, and that many other modifications would obvious to a skilled artisan in light of the teachings of the present disclosure.

Co-Therapy

Treatment of a patient with a solid tumor using a combination of the invention, such as an anti-CTLA-4 antibody, an anti-PD-L1 antibody, or antigen-binding fragments thereof, and an OX40 agonist or antigen-binding fragments thereof as provided herein can result in an additive or synergistic effect. As used herein, the term “synergistic” refers to a combination of therapies (e.g., a combination of anti-CTLA-4 antibody, anti-PD-L1 antibody, or antigen binding fragments thereof, and OX40 ligand fusion protein).

A synergistic effect of a combination of therapies (e.g., a combination of anti-CTLA-4 antibody, anti-PD-L1 antibody, or antigen binding fragments thereof, and OX40 ligand fusion protein) permits the use of lower dosages of one or more of the therapeutic agents and/or less frequent administration of said therapeutic agents to a patient with a solid tumor. The ability to utilize lower dosages of therapeutic agents and/or to administer said therapies less frequently reduces the toxicity associated with the administration of said therapies to a subject without reducing the efficacy of said therapies in the treatment of a solid tumor. In addition, a synergistic effect can result in improved efficacy of therapeutic agents in the management, treatment, or amelioration of an solid tumor. The synergistic effect of a combination of therapeutic agents can avoid or reduce adverse or unwanted side effects associated with the use of either single therapy.

In co-therapy, a combination of anti-CTLA-4 antibody, anti-PD-L1 antibody, or antigen binding fragments thereof, and OX40 ligand fusion protein can be optionally included in the same pharmaceutical composition, or may be included in one or more separate pharmaceutical compositions. In certain aspects, pharmaceutical compositions in accordance with the present disclosure comprise a pharmaceutically acceptable, non-toxic, sterile carrier such as physiological saline, non-toxic buffers, preservatives and the like. Suitable formulations for use in the treatment methods disclosed herein are described, e.g., in Remington's Pharmaceutical Sciences (Mack Publishing Co.) 16th ed. (1980).

Kits

The invention provides kits for enhancing anti-tumor activity. In one embodiment, the kit includes a therapeutic composition containing an anti-CTLA-4 antibody, anti-PD-L1 antibody, and OX40 agonist (e.g., an OX40 ligand fusion protein).

In some embodiments, the kit comprises a sterile container which contains a therapeutic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, the kit further comprises instructions for administering the therapeutic combinations of the invention. In particular embodiments, the instructions include at least one of the following: description of the therapeutic agent; dosage schedule and administration for enhancing anti-tumor activity; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLE

### Example 1

### Combination of OX40 Ligand Fusion Protein, Anti-CTLA-4 Antibody and Anti-PD-L1 Antibody Inhibited the Growth of a Cancer Cell Line in a Syngeneic Model

The antitumor activity of mOX40L FP (mouse OX40 ligand fusion protein), anti-PD-L1 (10F.9G2), and anti-CTLA-4 (9D9) was evaluated as monotherapy, as dual combination therapies, or as triple combination therapies in MCA205, a mouse syngeneic sarcoma model. Administration of mOX40L FP in combination with 10F.9G2 and 9D9 resulted in greater antitumor activity than administration of control articles or any of the above agents alone or in dual combination.

Test articles were obtained as follows: anti-CTLA-4 (9D9, BioXcell, West Lebanon, N.H.); anti-PD-L1 (10F.9G2, BioXcell, West Lebanon, N.H.); and OX40L FP (mouse OX40L fusion protein, MedImmune, Gaithersburg, Md.). MEDI6383, MEDI4736 and tremelimumab do not recognize mouse OX40, PD-L1 or CTLA-4, respectively. A murine OX40 ligand IgG1 fusion protein (mOX40L FP) was generated that binds to mouse OX40, triggers OX40 signaling, and was used as a surrogate mouse OX40 agonist for MEDI6383. 10F.9G2 is a commercially available rat IgG2b antibody against mouse PD-L1; and 9D9 is a commercially available mouse IgG2b antibody against mouse CTLA-4. As such, the effects and/or activities are expected to correspond to that in humans (e.g., when using human antibodies, amino acid sequences, etc.). Control articles were obtained as follows: OX40L FP Y182A (mouse OX40 ligand fusion protein having a Y to A amino acid substitution, MedImmune, Gaithersburg, Md.); mouse IgG2b isotype control (MPC-11, BioXcell, West Lebanon, N.H.); and rat IgG2b isotype control (MPC-11, BioXcell, West Lebanon, N.H.). Y182A mutant mouse OX40L mouse IgG1 fusion protein control comprises mOX40L FP with a single amino acid mutation at position 182 (Y to A amino acid change) in the receptor-binding domain, which prevents mOX40L binding to mouse OX40, but does not affect the overall structure of mOX40L. OX40L FP Y182A does not bind to native mouse or human OX40 and thus serves as a negative control for OX40L:OX40 interactions.

MCA205 syngeneic tumors were established in C57BL/6 mice as follows. MCA205 cells were obtained from Providence Cancer Center (Portland, Oreg.) and grown in RPMI 1640 medium (Roswell Park Memorial Institute 1640 medium, Life Technologies, Carlsbad, Calif.) supplemented with 10% fetal bovine serum (Life Technologies, Carlsbad, Calif.). MCA205 are chemically induced mouse soft tissue sarcoma tumor cells. Allografts were established by subcutaneous (SC) injection of 2.5×10⁵ MCA205 cells suspended in 0.1 mL of phosphate-buffered saline into the right flank of 7- to 9-week-old female C57BL/6 mice (Harlan Laboratories, Inc., Indianapolis, Ind.). C57BL/6 (total of 108) female mice were used in the study. C57BL/6 mice were randomly assigned after tumors grew to a mean volume of 185 mm³±1 mm³ per cohort, 11 days after implantation. Group designations, number of animals, dose levels, and dose schedule are presented at Table 1.

TABLE 1
Study Design: MCA205 Syngeneic Model

| | Number of | | Dose schedule | Dose level | |
|---|---|---|---|---|---|
| Group | animals (M/F) | Treatment | (study day) | (mg/kg)^{a} | ROA |
| 1 | 11 (F) | None | NA | NA | IP |
| 2 | 11 (F) | Isotype control mix | 11, 15, 18, 22 | 20 each | IP |
| 3 | 11 (F) | Anti-CTLA-4 mAb | 11, 15, 18, 22 | 20 | IP |
| 4 | 11 (F) | Anti-PD-L1 mAb | 11, 15, 18, 22 | 20 | IP |
| 5 | 11 (F) | mOX40L FP | 11, 15 | 20 | IP |
| 6 | 11 (F) | Anti-CTLA-4 mAb + Anti- | 11, 15, 18, 22; | 20 each | IP |
| | | PD-L1 mAb | 11, 15, 18, 22 | | |
| 7 | 11 (F) | Anti-CTLA-4 mAb + | 11, 15, 18, 22; | 20 each | IP |
| | | mOX40L FP | 11, 15 | | |
| 8 | 11 (F) | Anti-PD-L1 mAb + | 11, 15, 18, 22; | 20 each | IP |
| | | mOX40L FP | 11, 15 | | |
| 9 | 12 (F) | Anti-CTLA-4 mAb + Anti- | 11, 15, 18, 22; | 20 each | IP |
| | | PD-L1 mAb + mOX40L FP | 11, 15, 18, 22; | | |
| | | | 11, 15 | | |

F = female;
Isotype control mix contains clones MPC-11, LTF2 and mOX40L FP Y182A with Fc domains of mIgG2b, rIgG2b, and mIgG1 respectively;
IP = intraperitoneal;
M/F = male/female;
mAb = monoclonal antibody;
mOX40L FP = murine OX40 ligand murine IgG1 fusion protein;
NA = not applicable because the animals were not treated;
ROA = route of administration.
^{a}Dose volume: 0.2 mL.

All test articles and control articles were administered by intraperitoneal (IP) injection. There were no animal substitutions. The general health of mice was monitored daily for adverse clinical signs and bi-weekly for body weight. If hind limb paralysis, respiratory distress, 20% body weight loss, tumor volume greater than 2000 mm³ or ulcerated or necrotic tumors were noted, the animals were immediately sacrificed humanely by asphyxiation with CO₂. All experiments were conducted in accordance to AAALAC and MedImmune IACUC guidelines for humane treatment and care of laboratory animals.

Tumors were measured using a caliper thrice or twice weekly and tumor volumes were calculated using the following formula: tumor volume=[length (mm)×width (mm)²]/2 where length was defined as the larger side and width as the smaller side perpendicular to the length. Antitumor effects of each group were expressed as tumor growth inhibition (TGI), which was calculated as follows: percent TGI=(1−T/C)×100 where T=final tumor volumes from a treated group after the last dose and C=final tumor volumes from the control group after the last dose. Tumor growth responses were categorized as a complete response (CR) if there was no measureable tumor following treatment.

One-way ANOVA was used to determine mean tumor volume differences. In the event of a significant F test a Dunnett's or Sidak's multiple comparison test was utilized (where appropriate). Where applicable, a log 10 transformation was applied to tumor volumes to account for heteroscedasticity. A p value <0.05 was considered significant. Pairs of survival curves were compared using the logrank test. A Bonferroni correction was applied for multiple comparisons to control the familywise error rate. Prism 6.03 for Windows was used for the analysis. A P value <0.05 (unadjusted) was considered significant.

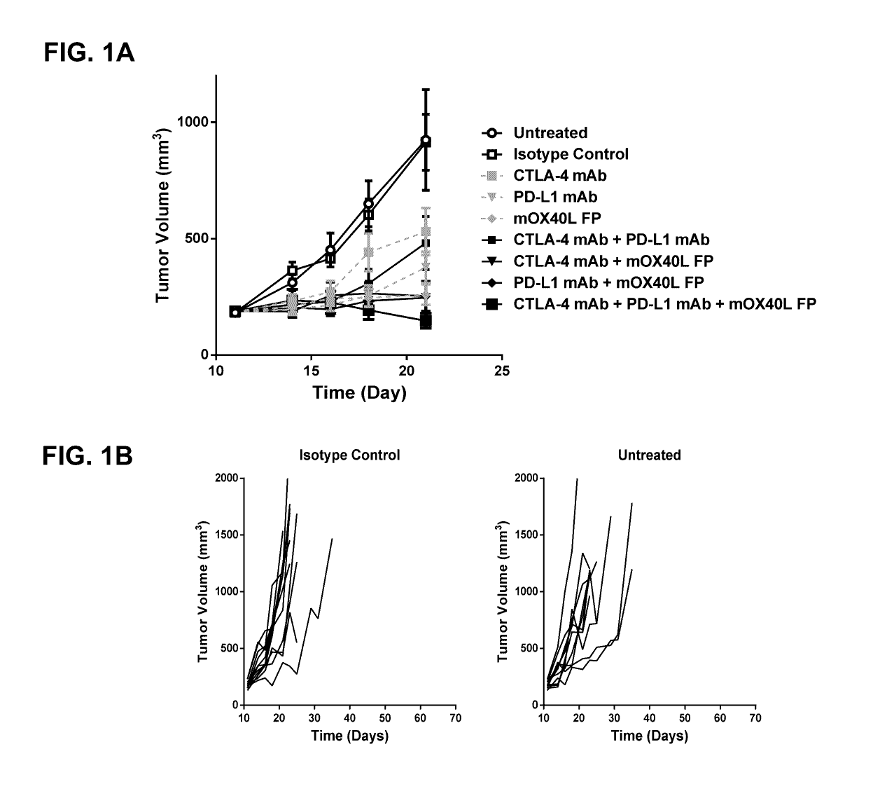

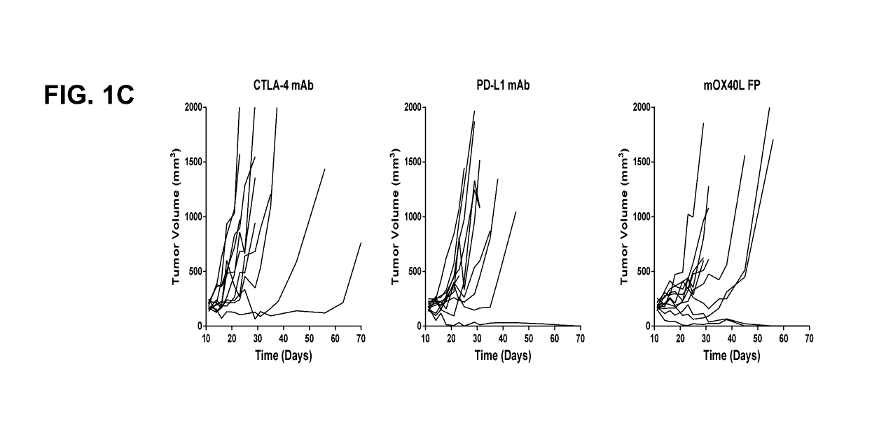

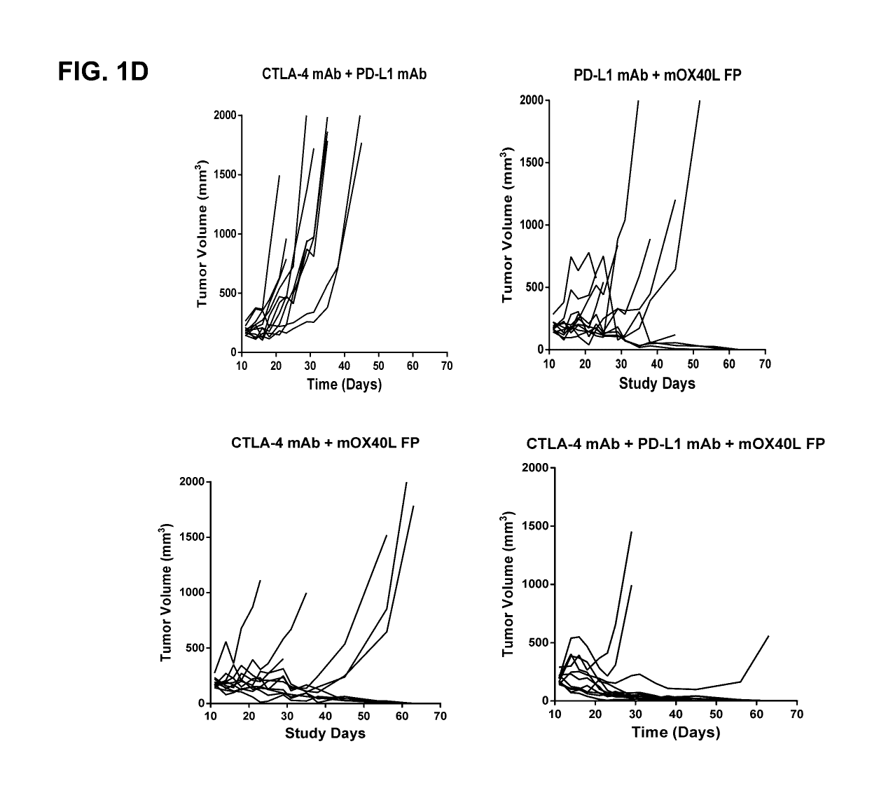

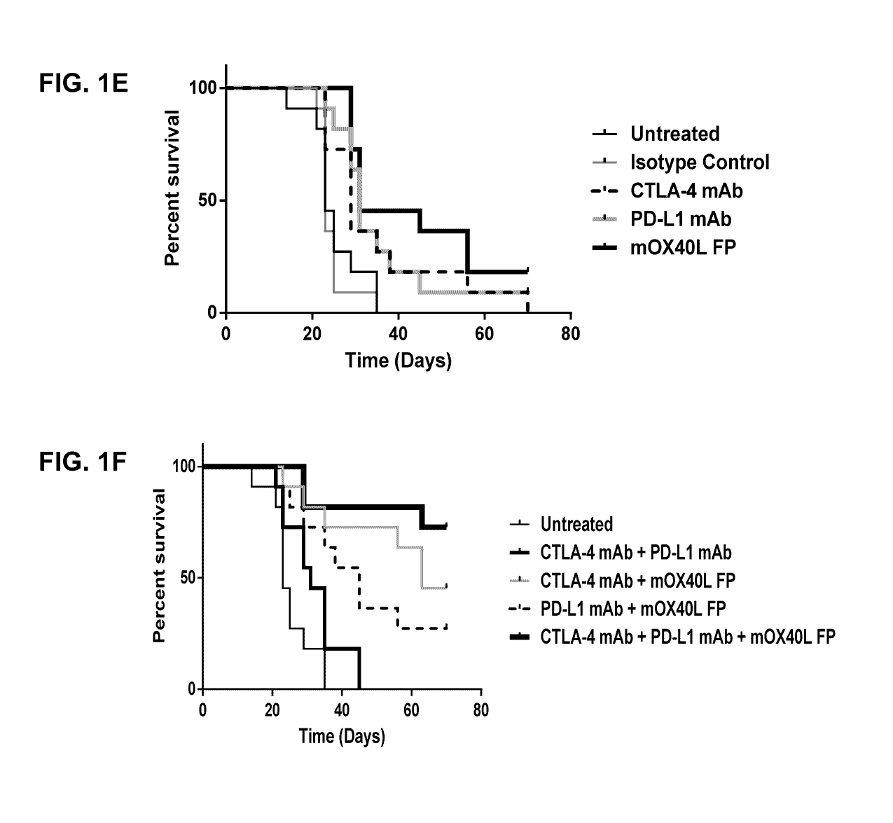

Treatment of groups of mice with mOX40L FP, anti-CTLA-4 mAb and anti-PD-L1 mAb as single agents resulted in reduced growth of MCA205 tumor cells compared to untreated and isotype control groups of mice (Table 2, FIG. 1A). Treatment of mice with anti-CTLA-4 mAb and anti-PD-L1 mAb in combination resulted in similar levels of tumor growth of MCA205 tumor cells compared to each of the respective agents alone (Table 2, FIG. 1A). Treatment of mice with mOX40L FP combined with anti-PD-L1 mAb or anti-CTLA-4 mAb reduced growth of MCA205 tumor cells compared to anti-PD-L1 mAb and anti-CTLA-4 mAb alone or to control groups but not compared to mOX40L FP alone (Table 2, FIG. 1A). Treatment of mice with mOX40L FP combined with anti-PD-L1 mAb and anti-CTLA-4 mAb reduced growth of MCA205 tumor cells compared to control groups, each therapeutic agent alone or combinations of two of any of the agents (Table 2, FIG. 1A).

TABLE 2
Treatment Groups, Percent TGI on Day 25, and Number of
Complete Responders in MCA205 Syngeneic Model

| | | | | Number of |
|---|---|---|---|---|
| | | | | Complete |
| | | | | Responders out of |
| Group^{a} | Test/Control Article | Dose^{b} (mg/kg) | % TGI^{c} | 11 mice^{d} |
| 1 | None | NA | NA | 0 |
| 2 | Isotype control mix | 20 mg/kg | NA | 0 |
| 3 | Anti-CTLA-4 mAb | 20 mg/kg | 42 | 0 |
| 4 | Anti-PD-L1 mAb | 20 mg/kg | 59 | 1 |
| 5 | mOX40L FP | 20 mg/kg | 72 | 2 |
| 6 | Anti-CTLA-4 mAb + Anti-PD-L1 mAb | 20 mg/kg each | 47 | 0 |
| 7 | Anti-CTLA-4 mAb + mOX40L FP | 20 mg/kg each | 73 | 5 |
| 8 | Anti-PD-L1 mAb + mOX40L FP | 20 mg/kg each | 72 | 3 |
| 9 | Anti-CTLA-4 mAb + Anti-PD-L1 mAb + | 20 mg/kg each | 84 | 8 |
| | mOX40L FP | | | |

IP = intraperitoneal;
NA = not applicable;
TGI = tumor growth inhibition;
V = volume.
^{a}n = 11
^{b}All animals received 200 μL of test articles IP on Days 11 and 15 for mOX40L FP and on Days 11, 15, 18 and 22 for anti-CTLA-4 mAb and anti-PD-L1 mAb.
^{c}% TGI = [1 − (mean tumor V of treatment group) ÷ (mean tumor V of isotype control group)] × 100
^{d}Number of animals in a group with a tumor volume measurement recorded as zero at the end of the study.

Heterogeneity of response between individual animals is often observed in syngeneic models. However, the increased response to mOX40L FP, anti-CTLA-4 mAb and anti-PD-L1 mAb, when used in combination therapy, was evident from the individual animal tumor growth graphs (FIGS. 1B-1D). Untreated and isotype control treated animals were euthanized by Day 45 due to large tumor sizes (FIG. 1B).

Administration of anti-CTLA-4 mAb in combination with anti-PD-L1 mAb resulted in no increase in antitumor activity (as defined by percentage TGI and number of CRs) relative to treatment with either antibody alone as monotherapy at the same dose level (Table 2, FIG. 1C). Complete responses were observed in 0 of 11 mice in the combination group, compared with 0 of 11 mice treated with anti-CTLA-4 mAb and 1 of 11 treated with anti-PD-L1 mAb alone (Table 2).

Administration of anti-CTLA-4 mAb in combination with mOX40L FP resulted in similar antitumor activity (as defined by a similar percentage TGI and the number of CRs) relative to treatment with mOX40L FP alone as monotherapy at the same dose level (Table 2, FIG. 1D); greater antitumor activity (as defined by percentage TGI and the number of CRs) relative to treatment with anti-CTLA-4 mAb alone as monotherapy at the same dose level (Table 2, FIG. 1D). Complete responses were observed in 5 of 11 mice in the combination group, compared with 0 of 11 treated with anti-CTLA-4 mAb and 2 of 11 mice treated with mOX40L FP alone (Table 2).

Administration of anti-CTLA-4 mAb and anti-PD-L1 mAb together in combination with mOX40L FP resulted in increased antitumor activity (as defined by a higher TGI and a greater number of CRs) relative to treatment with the agents alone as monotherapy or dual therapy combinations each at the same dose level (Table 2, FIG. 1D). Complete responses were observed in 8 of 11 mice in the triple combination group, compared with 0 of 11 treated with anti-CTLA-4/PD-L1 mAb combination, 5 of 11 mice treated with anti-CTLA-4/mOX40L FP combo, and 3 of 11 mice treated with anti-PD-L1/mOX40L FP combo (Table 2).

None of the untreated mice or mice administered the isotype control or anti-CTLA-4 mAb survived until the end of the study with the median survival time of mice was 23 days, 23 days, and 29 days respectively (FIG. 1E). Administration of anti-PD-L1 mAb or mOX40L FP resulted in increased median survival time to 31 days for each agent. One of 11 mice and two of 11 mice survived until the end of the study on Day 70 following administration of anti-PD-L1 mAb and mOX40L FP, respectively (FIG. 1E).

Administration of anti-CTLA-4 mAb in combination with anti-PD-L1 mAb did not result in increased activity relative to treatment with either antibody alone when used as monotherapy at the same dose level (FIG. 1F). Median survival time in was 31 days as compared to 29 days (anti-CTLA-4 mAb) and 31 days (anti-PD-L1 mAb). No animals survived until the end of the study.

Administration of anti-CTLA-4 mAb in combination with mOX40L FP resulted in increased activity relative to treatment with either antibody alone when used as monotherapy at the same dose level (FIG. 1F). Median survival time was 45 days as compared to 31 days (anti-PD-L1 mAb) and 31 days (mOX40L FP). In addition, 3 of 11 mice in the anti-PD-L1 mAb/mOX40L FP combination group survived until the end of the study, as compared to 1 of 11 mice (anti-PD-L1 mAb) and 2 of 11 mice (mOX40L FP).

Administration of anti-CTLA-4 in combination with anti-PD-L1 mAb and mOX40L FP resulted in increased activity relative to treatment with either agent alone or with dual combinations of the agents at the same dose level (FIG. 1F). Due to the number of animals that survived until the end of the study, it was not possible to calculate median survival time for the triple combination group. In addition, 8 of 11 animals in the triple combination group survived until the end of the study, as compared to (pairwise comparisons of the survival curves using the Bonferonni adjustment for multiple comparisons) 0 of 11 treated with anti-CTLA-4/PD-L1 mAb combination (p=0.0002; significant), 5 of 11 mice treated with anti-CTLA-4/mOX40L FP combination (p>0.05; not significant), and 3 of 11 mice treated with anti-PD-L1/mOX40L FP combination (p=0.21; not significant) (FIG. 1F).

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### SEQUENCE LISTING

MEDI4736 VL
>PCT/US2010/058007_77 Sequence 77 from PCT/US2010/
058007 Organism: Homo sapiens
SEQ ID NO: 1
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIK
MEDI4736 VH
>PCT/US2010/058007_72 Sequence 72 from PCT/U_2010/
058007 Organism: Homo sapiens
SEQ ID NO: 2
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSS
MEDI4736 VH CDR1
>PCT/US2010/058007_73 Sequence 73 from PCT/US2010/
058007 Organism: Homo sapiens
SEQ ID NO: 3
RYWMS
MEDI4736 VH CDR2
>PCT/US2010/058007_74 Sequence 74 from PCT/US2010/
058007 Organism: Homo sapiens
SEQ ID NO: 4
NIKQDGSEKYYVDSVKG
MEDI4736 VH CDR3
>PCT/US2010/058007_75 Sequence 75 from PCT/US2010/
058007 Organism: Homo sapiens
SEQ ID NO: 5
EGGWFGELAFDY
MEDI4736 VL CDR1
>PCT/US2010/058007_78 Sequence 78 from PCT/US2010/
058007 Organism: Homo sapiens
SEQ ID NO: 6
RASQRVSSSYLA
MEDI4736 VL CDR2
>PCT/US2010/058007_79 Sequence 79 from PCT/US2010/
058007 Organism: Homo sapiens
SEQ ID NO: 7
DASSRAT
MEDI4736 VL CDR3
>PCT/US2010/058007_80 Sequence 80 from PCT/US2010/
058007 Organism: Homo sapiens
SEQ ID NO: 8
QQYGSLPWT
Tremelimumab
>SEQ ID NO: 22 from US 6,682,736
SEQ ID NO: 9
PSSLSASVGDRVTITCRASQSINSYLDWYQQKPGKAPKLLIYAASSLQSG
VPSRFSGSGSGTDFTLTISSLQPEDFATYYCQQYYSTPFTFGPGTKVEIK
RTVAAPSVFIFPPSDEQLKSGTASVVCLLNNFYPREAKV
Tremelimumab VH
>SEQ ID NO:9 from US 6,682,736
SEQ ID NO: 10
GVVQPGRSLRLSCAASGFTFSSYGMHWVRQAPGKGLEWVAVIWYDGSNKY
YADSVKGRFTISRDNSKNTLYLQMNSLRAEDTAVYYCARDPRGATLYYYY
YGMDVWGQGTTVTVSSASTKGPSVFPLAPCSRSTSESTAALGCLVKDYFP
EPVTVSWNSGALTSGVH
Tremelimumab VH CDR1
SEQ ID NO: 11
GFTFSSYGMH
Tremelimumab VH CDR2
SEQ ID NO: 12
VIWYDGSNKYYADSV
Tremelimumab VH CDR3
SEQ ID NO: 13
DPRGATLYYYYYGMDV
Tremelimumab VL CDR1
SEQ ID NO: 14
RASQSINSYLD
Tremelimumab VL CDR2
SEQ ID NO: 15
AASSLQS
Tremelimumab VL CDR3
SEQ ID NO: 16
QQYYSTPFT
MEDI6383 OX40 Agonist
SEQ ID NO: 17
ESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGKDQDKIEALSSKVQQLERSIGL
KDLAMADLEQKVLEMEASTQVSHRYPRIQSIKVQFTEYKKEKGFILTSQK
EDEIMKVQNNSVIINCDGFYLISLKGYFSQEVNISLHYQKDEEPLFQLKK
VRSVNSLMVASLTYKDKVYLNVTTDNTSLDDFHVNGGELILIHQNPGEFC
VL

What is claimed is:

1. A method of treating a solid tumor in a subject, comprising administering an anti-PD-L1 antibody or an antigen-binding fragment thereof, an anti-CTLA-4 antibody or an antigen-binding fragment thereof, and an OX40 agonist to the subject.

2. The method of claim 1, wherein the OX40 agonist is one or more of an OX40 ligand fusion protein or anti-OX40 antibody.

3. The method of claim 2, wherein the OX40 ligand fusion protein is MEDI6383.

4. The method of claim 1, wherein the anti-PD-L1 antibody is MEDI4736.

5. The method of claim 1, wherein the anti-CTLA-4 antibody is tremelimumab.

6. A method of treating a solid tumor in a subject, comprising administering MEDI4736 or an antigen-binding fragment thereof, tremelimumab or an antigen-binding fragment thereof, and MEDI6383 to the subject.

7. The method of claim 6, wherein the administration increases survival.

8. The method of claim 7, wherein the administration results in an increase in survival as compared to the administration of MEDI4736 alone, tremelimumab alone, or MED6383 alone.

9. The method of claim 7, wherein the administration results in an increase in survival as compared to the administration of MEDI4736 and tremelimumab, MEDI4736 and MEDI6383, and tremelimumab and MEDI6383.

10. The method of claim 9, wherein the administration decreases tumor volume.

11. The method of claim 10, wherein the administration results in a decrease in tumor volume as compared to the administration of MEDI4736 alone, tremelimumab alone, or MED6383 alone.

12. The method of claim 10, wherein the administration results in a decrease in tumor volume as compared to the administration of MEDI4736 and tremelimumab, MEDI4736 and MEDI6383, and tremelimumab and MEDI6383.

13. The method of claim 1, wherein the administration of the anti-PD-L1 antibody or an antigen-binding fragment thereof is by intravenous infusion.

14. The method of claim 1, wherein the administration of the anti-CTLA-4 antibody or an antigen-binding fragment thereof is by intravenous infusion.

15. The method of claim 1, wherein the administration of MEDI6383 or active fragment thereof is by intravenous infusion.

16. The method of claim 1, wherein the solid tumor is an ovarian cancer, breast cancer, colorectal cancer, prostate cancer, cervical cancer, uterine cancer, testicular cancer, bladder cancer, head and neck cancer, melanoma, pancreatic cancer, renal cell carcinoma, or lung cancer.

17. The method of claim 16, wherein the solid tumor is triple negative breast cancer.

18. The method of claim 17, wherein the solid tumor is a non-small cell lung cancer.

19. The method of claim 18, wherein the solid tumor is squamous or non-squamous non-small cell lung cancer.

20. The method of claim 1, wherein the subject is a human patient.

21. A pharmaceutical composition comprising an effective amount of an anti-PD-L1 antibody or an antigen-binding fragment thereof, an anti-CTLA-4 antibody or an antigen-binding fragment thereof, and an OX40 agonist and a pharmaceutically acceptable excipient.

22. The pharmaceutical composition of claim 21, wherein the OX40 agonist is MEDI6383.

23. The pharmaceutical composition of claim 21, wherein the anti-PD-L1 antibody is MEDI4736.

24. The pharmaceutical composition of claim 21, wherein the anti-CTLA-4 antibody is tremelimumab.

25. A pharmaceutical composition comprising an effective amount of MEDI4736 or an antigen-binding fragment thereof, tremelimumab or an antigen-binding fragment thereof, and MEDI6383 and a pharmaceutically acceptable excipient.

26. The pharmaceutical composition of claim 21 formulated for intravenous administration.


SEQUENCE LISTING (28 sequences)

SEQ ID NO: 1 - protein, 108 aa, Homo sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, Homo sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 5 aa, Homo sapiens
RYWMS

SEQ ID NO: 4 - protein, 17 aa, Homo sapiens
NIKQDGSEKYYVDSVKG

SEQ ID NO: 5 - protein, 12 aa, Homo sapiens
EGGWFGELAFDY

SEQ ID NO: 6 - protein, 12 aa, Homo sapiens
RASQRVSSSYLA

SEQ ID NO: 7 - protein, 7 aa, Homo sapiens
DASSRAT

SEQ ID NO: 8 - protein, 9 aa, Homo sapiens
QQYGSLPWT

SEQ ID NO: 9 - protein, 139 aa, Artificial Sequence
  Description of Artificial Sequence Synthetic polypeptide
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 10 - protein, 167 aa, Artificial Sequence
  Description of Artificial Sequence Synthetic polypeptide
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 11 - protein, 10 aa, Artificial Sequence
  Description of Artificial Sequence Synthetic peptide
GFTFSSYGMH

SEQ ID NO: 12 - protein, 15 aa, Artificial Sequence
  Description of Artificial Sequence Synthetic peptide
VIWYDGSNKYYADSV

SEQ ID NO: 13 - protein, 16 aa, Artificial Sequence
  Description of Artificial Sequence Synthetic peptide
DPRGATLYYYYYGMDV

SEQ ID NO: 14 - protein, 11 aa, Artificial Sequence
  Description of Artificial Sequence Synthetic peptide
RASQSINSYLD

SEQ ID NO: 15 - protein, 7 aa, Artificial Sequence
  Description of Artificial Sequence Synthetic peptide
AASSLQS

SEQ ID NO: 16 - protein, 9 aa, Artificial Sequence
  Description of Artificial Sequence Synthetic peptide
QQYYSTPFT

SEQ ID NO: 17 - protein, 402 aa, Artificial Sequence
  Description of Artificial Sequence Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDQDKIEALSSKVQQLERSIGLKDLAMADLEQKVLEMEASTQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 18 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 19 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 20 - protein, 410 aa, Artificial Sequence [sequence omitted]

SEQ ID NO: 21 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 22 - protein, 107 aa, Artificial Sequence
  Description of Artificial Sequence Synthetic polypeptide
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIK

SEQ ID NO: 23 - protein, 107 aa, Artificial Sequence
  Description of Artificial Sequence Synthetic polypeptide
DIQMTQSPSSLSASVGDRVTITCRASQDISNYLNWYQQKPGKAVKLLIYYTSKLHSGVPSRFSGSGSRTDYTLTISSLQPEDFATYYCQQGSALPWTFGQGTKVEIK

SEQ ID NO: 24 - protein, 121 aa, Artificial Sequence
  Description of Artificial Sequence Synthetic polypeptide
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSS

SEQ ID NO: 25 - protein, 451 aa, Artificial Sequence
  Description of Artificial Sequence Synthetic polypeptide
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 214 aa, Artificial Sequence
  Description of Artificial Sequence Synthetic polypeptide
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 412 aa, Artificial Sequence
  Description of Artificial Sequence Synthetic polypeptide
APLATDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKELLGGGSIKQIEDKIEEILSKIYHIENEIARIKKLIGERGHGGGSNSQVSHRYPRFQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 28 - protein, 15 aa, Artificial Sequence
  Description of Artificial Sequence Synthetic peptide
GGGGSGGGGSGGGGS